(19) United States
(12) Patent Application Publication
(10) Pub. No.: US 2011/0003742 A1
(43) Pub. Date: Jan. 6, 2011

(54) Naphthalimide Dosing by N-Acetyl Transferase Genotyping

(75) Inventor: Dennis M. Brown, Menlo Park, CA (US)

Correspondence Address: MORGAN, LEWIS & BOCKIUS, LLP (SF), ONE MARKET SPEAR STREET TOWER, SAN FRANCISCO, CA, 94105, US

(73) Assignee: ChemGenex Pharmaceuticals, Inc., Menlo Park, CA (US)

(21) Appl. No.: 12/720,515

(22) Filed: Mar. 9, 2010

Related U.S. Application Data
(63) Continuation of application No. 11/048,614, filed on Jan. 31, 2005, now abandoned.
(60) Provisional application No. 60/540,805, filed on Jan. 30, 2004.

(51) Int. Cl. A61K 38/18; A61K 31/473; A61P 35/00; C12Q 1/68

(52) U.S. Cl. 514/7.6; 514/296; 435/6

(57) ABSTRACT

The present invention provides methods for dosing patients with naphthalimides, including amonafide, amonafide salts, and analogs thereof based on N-acetyl transferase genotyping. The invention also provides methods for dosing the amount of granulocyte colony stimulating factor (GCSF) used in combination with naphthalimide to prevent or modulate leukocytopenia.

This application is a continuation of U.S. patent application Ser. No. 11/048,614, filed Jan. 31, 2005, which claims the benefit of U.S. Provisional Application No. 60/540,805, filed Jan. 30, 2004.

### FIELD OF THE INVENTION

The present invention provides methods for dosing patients with naphthalimides, including amonafide, amonafide salts, and analogs thereof based on N-acetyl transferase genotyping. The invention also provides methods for dosing the amount of granulocyte colony stimulating factor (GCSF) used in combination with naphthalimide to prevent or modulate leukocytopenia in a patient.

### BACKGROUND OF THE INVENTION

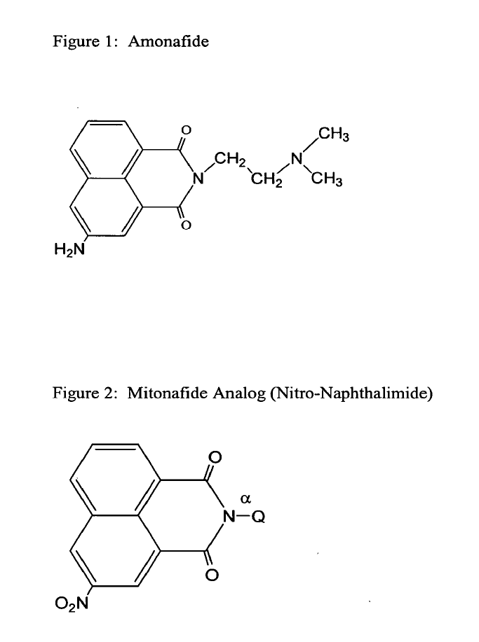

Amonafide, a member of the naphthalimide family, is a known antitumor compound. Its structure is shown in FIG. 1. Although amonafide has antitumor activity, it has not been approved for use in human chemotherapy because of unpredictable toxicity.

It is an object of the invention to provide methods for determining naphthalimide dosages to minimize leukocytopenia in patients.

### SUMMARY OF THE INVENTION

The invention includes methods for dosing a patient with naphthalimide. The method comprises genotyping a patient for an N-acetyl transferase genotype to provide an indication of the phenotype of said patient to rapidly or slowly acylate naphthalimide. Based on the phenotype, the dose of naphthalimide to be administered is determined. A higher amount of naphthalimide is administered to a patient with a slow acylation phenotype as compared to a fast acylator phenotype. A lower dose of the naphthalimides administered to a patient with a fast acylation phenotype as compared to a slow acylator phenotype.

In some embodiments, the fast or slow acylation phenotype is also used to determine the dose of granulocyte colony stimulating factor (GCSF) to be administered to treat leukocytopenia associated with the administration of the naphthalimide. The GCSF may be administered contemporaneously with, prior to or after the administration of the naphthalimide.

In other embodiments, the method comprises administering an anti-proliferative agent to the patient before, during or after the administration of either the naphthalimide and/or the GCSF.

The invention includes a composition comprising a naphthalimide and GCSF. In some embodiments, the composition further comprises an additional anti-proliferative agent. The invention also includes kits for N-acetyl transferase genotyping comprising at least one nucleic acid capable of distinguishing at least one allele of a genotype of N-acetyl transferase; and at least one reagent or label required for the genotyping assay. In another embodiment, the reagents include a DNA polymerase for carrying out a polymerase chain reaction (PCR).

### DETAILED DESCRIPTION OF THE FIGURES

FIG. 1 depicts the structure of the naphthalimide, amonafide.

FIG. 2 depicts the structure of a 3-nitro-naphthalimide or mitonafide analog.

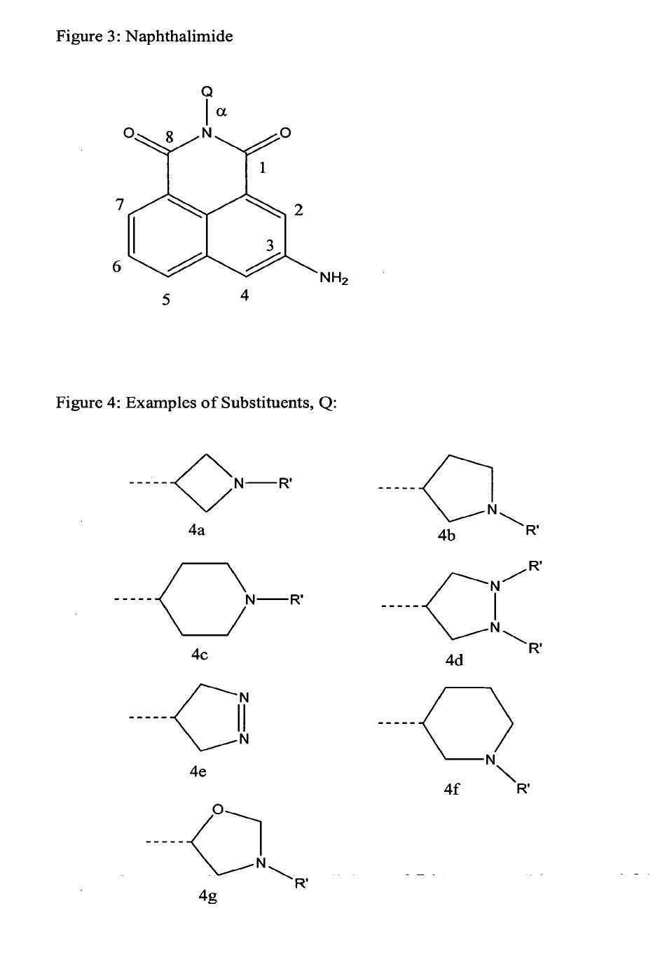

FIG. 3 depicts the structure of a naphthalimide. The Q in the figure represents a substituent group, as described herein.

FIG. 4 depicts chemical structures of several possible Q substituent groups that may substitute in the naphthalimide structure of FIG. 3, or in the nitro-naphthalimide structure of FIG. 2. The ring structures each depict a bond to the amide nitrogen. This bond (marked by a dashed line) represents the point of attachment to the naphthalimide structure of FIG. 3 or to the nitro-naphthalimide structure of FIG. 2.

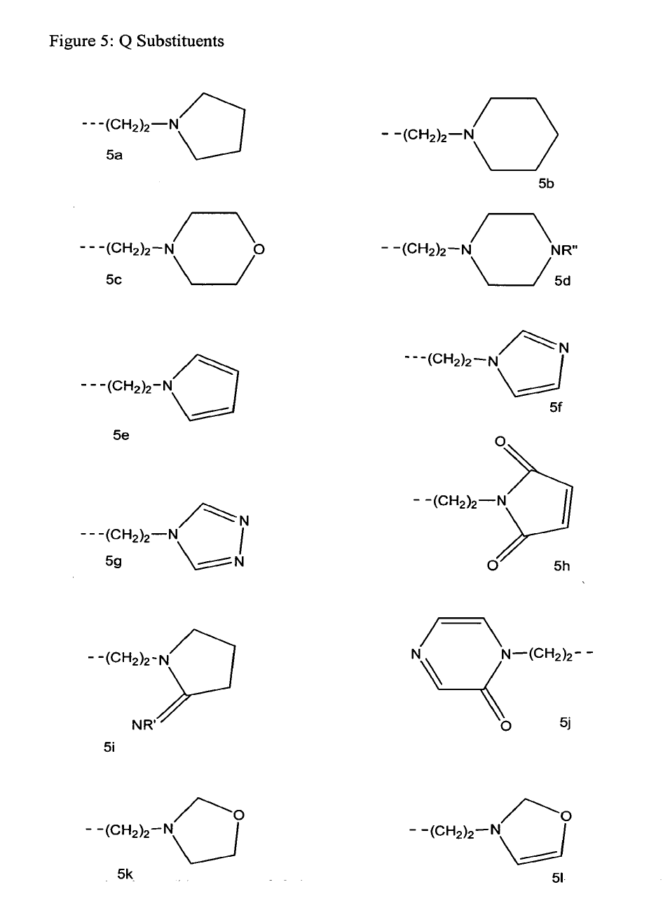

FIG. 5 depicts another groups of possible Q substituent groups. Similar to those of FIG. 4, these groups may substitute for Q in the naphthalimide structure of FIG. 3, or in the nitro-naphthalimide structure of FIG. 2. Each structure depicts a bond (marked by a dashed line), which represents the point of attachment of the substituent group to the naphthalimide structure of FIG. 3 or to the nitro-naphthalimide structure of FIG. 2.

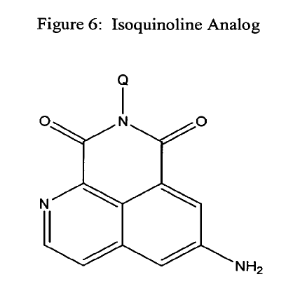

FIG. 6 depicts the structure of an isoquinoline analog of amonafide. The Q in the figure represents a substituent group, as described herein.

### DETAILED DESCRIPTION OF THE INVENTION

Naphthalimides, such as amonafide, are metabolically processed. The first step of metabolism is to acylate the naphthalimide by way of N-acetyl transferase (NAT). In one aspect, the invention utilizes an assay to genotype a patient to determine whether he falls within one of two phenotypes: (1) slow acylators of naphthalimide or (2) fast acylators which include either the rapid (R) homozygous or intermediate (I) genotype. In most cases, the fast phenotype includes heterozygous genotypes of rapid and intermediate NAT-2 genes. See D. W. Hein, et al., “Molecular Genetics and Epidemiology of the NAT1 and NAT2 Acetylation Polymorphisms,” Cancer Epidemiology, Biomarkers & Prevention Vol. 9, 29-42, January 2000.

Acetyl naphthalimide, in general, has significant anti-tumor activity. However, the acylated naphthalimide, e.g., acetyl amonafide, has a profound impact upon the white blood cell count (WBC) of the patient. In particular, acetyl amonafide has been shown to induce leukocytopenia and, in particular, granulocytopenia. By genotyping a patient prior to treatment, it is possible to determine the dosage levels and intervals of naphthalimide administration so as to minimize leukocytopenia, thereby controlling a significant toxic side effect. For example, a slow acylator will have lower levels of acylated naphthalimide and a lower ratio of acylated naphthalimide to naphthalimide as compared to a fast acylator. Such patients are least likely to present a severe leukocytopenia. Accordingly, such patients can tolerate an increase in the normal dosage of the naphthalimide for treatment. On the other hand, a fast acylator will have a higher level of acylated naphthalimide and a higher ratio of acylated naphthalimide as compared to naphthalimide. Such patients are more likely to present a significant leukocytopenia upon treatment with naphthalimide. In such cases, the dose of the naphthalimide can be decreased based on the genotype prior to administration so as to reduce the likelihood of severe leukocytopenia.

For example, the dosage of a naphthalimide such as amonafide, for a slow acylator would be in the range of 300-1000 mg/m.sup.2, more preferably between 400 and 600 mg/m.sup.2, and most preferably between 450 and 550 mg/m.sup.2. In the case of the fast acylator, naphthalimide dosages would be reduced to between 50 and 450 mg/m.sup.2, more preferably between 150 and 450 mg/m.sup.2, and most preferably between 350 and 450 mg/m.sup.2. In the case of the fast acylator, these dosages can be increased when used in conjunction with GCSF and may be as high as the dosage for the slow acylator.

In addition to the foregoing, the fast or slow acylator genotype of the patient may also be used to dose the patient with anti-leukocytopenia agents such as granulocyte colony stimulating factor (GCSF) also referred to as Neupogen.RTM. from Amgen, Thousand Oaks, Calif. Accordingly, higher doses of GCSF are called for in case of fast acylators that are treated with naphthalimide. Alternatively, in the case of slow acylators, the dosage of GCSF can be reduced or eliminated entirely in the naphthalimide treatment regime.

The use of genotyping patients prospectively to identify fast and slow acylator phenotypes provides the opportunity to selectively employ GCSF, to boost neutrophil counts for patients at greater risk for neutropenia (e.g., rapid acylators can be dosed above 300 mg/m.sup.2/week). In these cases, the potential for increased naphthalimide doses may be boosted if the GCSF maintains relatively normal leucocyte levels. In addition, for slow acylators, the opportunity to increase naphthalimide doses above, for example, 600 mg/m.sup.2/week may also exist if GCSF can be used.

With the identification of a patient's NAT-2 genotype, GCSF therapy may be initiated prior to the initiation of naphthalimide in an effort to increase leucocyte count to prevent the myelosuppressive effects of the naphthalimide. The GCSF, for example, administered either I.V. or S.C. at doses ranging from 3-10 mcg/kg given daily could boost the leucocyte count such that vulnerable rapid acylators could safely receive the established doses for that phenotype but may allow for the opportunity to increase naphthalimide dosages and/or the frequency of dosing (e.g., daily, two times per week, etc.).

The same opportunity may also exist for slow acylators where ultra high dosing (e.g., >650 mg/m.sup.2) may be achieved with GCSF supportive therapy.

As used herein, naphthalimide includes all members of that chemical family including benz isoquinoline dione and analogs thereof. Naphthalimides have the structure set forth in FIG. 3. In addition, the naphthalimide includes amonafide such as set forth in FIG. 1 and analyzed thereof. Naphthalimide also includes nitro-naphthalimide, e.g., mitonafide as set forth in FIG. 2 and analogs such as isoquinoline analogs such as set forth in FIG. 6. It should be understood that Q in each of the FIGS. 2 and 6 correspond to the structure set forth in FIG. 4 as well as other substituents at Q.

A patient for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

Naphthalimides can be used to treat a cellular proliferative disease. According to a preferred embodiment, the cellular proliferative disease is a tumor, e.g., a solid tumor. Solid tumors that are particularly amenable to treatment by the claimed methods include carcinomas and sarcomas. Carcinomas include those malignant neoplasmas derived from epithelial cells which tend to infiltrate (invade) the surrounding tissues and give rise to metastases. Adenocarcinomas are carcinomas derived from glandular tissue or in which the tumor cells form recognizable glandular structures. Sarcomas broadly include tumors whose cells are embedded in a fibrillar or homogeneous substance like embryonic connective tissue.

It will be understood that the method of the invention is not limited to the treatment of these tumor types, but extends to any solid tumor derived from any organ system.

Cellular proliferative diseases that can be treated by naphthalimide include, for example, psoriasis, skin cancer, viral induced hyperproliferative HPV-papiloma, HSV-shingles, colon cancer, bladder cancer, breast cancer, melanoma, ovarian carcinoma, prostatic carcinoma, or lung cancer, and a variety of other cancers as well.

Naphthalimides are provided in a dosage amount (1) sufficient to modulate a cellular proliferative disease and (2) minimize leukocytopenia. In one embodiment, modulation of a cellular proliferative disease comprises a reduction in tumor growth. In another embodiment, modulation of a disease comprises inhibition of tumor growth. In another embodiment, modulation of a cellular proliferative disease comprises an increase in tumor volume quadrupling time (described below). In another embodiment, modulation of a cellular proliferative disease comprises a chemopotentiator effect. In another embodiment, modulation of a disease comprises a chemosensitizing effect. In other embodiments, modulation of a disease comprises cytostasis. In still other embodiments, modulation of a disease comprises a cytotoxic effect.

Naphthalimides are administered to a host by a variety of routes. According to one embodiment, a naphthalimide is administered by injection, preferably by parenteral, e.g., intravenous, injection. According to one embodiment, an antiproliferative agent is administered by injection, preferably by intravenous injection. The mode of administration of the agents may be the same or different for each. Thus, the compounds may be administered in a single dosage form, one may be administered orally and the other intravenously, one may be administered continuously and the other intermittently, etc.

Any suitable route of administration may be employed for providing a mammal, especially a human, with an effective dosage of the compounds of the invention. For example, oral, rectal, topical, parenteral, ocular, pulmonary, nasal, etc. routes may be employed. Dosage forms include tablets, troches, dispersions, suspensions, solutions, capsules, creams, ointments, aerosols, and the like. The routes of administration may be the same or different for each of the two compounds.

Disclosed herein are methods of treatment comprising contacting a host with a naphthalimide in conjunction with GCSF alone or further in conjunction with an antiproliferative agent. By “in conjunction with” is meant that the agents are administered such that the agents are present and active in the host together during at least a portion of the treatment schedule. According to one embodiment, two or more agents are administered simultaneously, in a single dosage form.

According to an alternative embodiment, the administration of one agent is followed by administration of the other agent. For example, administration of a naphthalimide may be followed by administration of an GCSF agent; or administration of GCSF may be followed by administration of a naphthalimide.

When administration of the two agents is not simultaneous, a defined length of time may separate the two agents. According to one embodiment, administration of each agent is separated by at least about 5 minutes but by no more than 4 hours. Generally, when administration of the two agents is not simultaneous, the time separating the administration of each agent is no more than two plasma half lives of the first administered agent. According to a preferred embodiment, administration of each agent is separated by about 30 minutes. According to another embodiment, administration of each agent is separated by about 1 hour. According to another embodiment, administration of each agent is separated by about 2 hours.

The optimal time separating the administration of the agents will vary depending on the dosage used, the clearance rate of each agent, and the particular host treated. According to the claimed methods, the naphthalimide GCSF and alternatively an antiproliferative agent used are administered such that the agents are present together in the host system in active form during the treatment of the host. That is, the agent that is administered first will be present in the host in an active form after the second agent is administered.

A chemical agent is a “chemopotentiator” when it enhances the effect of a known antiproliferative drug in a more than additive fashion relative to the activity of the chemopotentiator or antiproliferative agent used alone. In some cases, a “chemosensitizing” effect may be observed. This is defined as the effect of use of an agent that if used alone would not demonstrate significant antitumor effects but would improve the antitumor effects of an antiproliferative agent in a more than additive fashion than the use of the antiproliferative agent by itself.

As used herein, antiproliferative agents are compounds which induce cytostasis or cytotoxicity. “Cytostasis” is the inhibition of cells from growing while “cytotoxicity” is defined as the killing of cells.

Specific examples of antiproliferative agents include: antimetabolites, such as methotrexate, 5-fluorouracil, gemcitabine, cytarabine, pentostatin, 6-mercaptopurine, 6-thioguanine, L-asparaginase, hydroxyurea, N-phosphonoacetyl-L-aspartate (PALA), fludarabine, 2-chlorodeoxyadenosine, and floxuridine; structural protein agents, such as the vinca alkaloids, including vinblastine, vincristine, vindesine, vinorelbine, paclitaxel, and colchicine; agents that affect NF-.kappa.B, such as curcumin and parthenolide; agents that affect protein synthesis, such as homoharringtonine; antibiotics, such as dactinomycin, daunorubicin, doxorubicin, idarubicin, bleomycins, plicamycin, and mitomycin; hormone antagonists, such as tamoxifen and luteinizing hormone releasing hormone (LHRH) analogs; nucleic acid damaging agents such as the alkylating agents mechlorethamine, cyclophosphamide, ifosfamide, chlorambucil, dacarbazine, methylnitrosourea, semustine (methyl-CCNU), chlorozotocin, busulfan, procarbazine, melphalan, carmustine (BCNU), lomustine (CCNU), and thiotepa, the intercalating agents doxorubicin, dactinomycin, daurorubicin and mitoxantrone, the topoisomerase inhibitors etoposide, camptothecin and teniposide, and the metal coordination complexes cisplatin and carboplatin.

Pharmaceutical compositions comprise a naphthalimide and GCSF. They may further comprise an antiproliferative agent. The naphthalimide and GCSF or naphthalimide, GCSF and antiproliferative agent may be in intimate admixture or they may isolated from each other. In some embodiments, the naphthalimide in the pharmaceutical compositions is a pharmaceutically acceptable salt. Accordingly, pharmaceutical compositions may contain pharmaceutically acceptable carriers and, optionally, other therapeutically active ingredients.

The agents may be provided in a range of concentrations, depending on the cellular proliferative disease to be treated, host species, clearance rate of each agent, drug absorption, bioavailability, mode of administration. In a preferred embodiment, a naphthalimide is provided for administration at between about 1-30 mg/kg or 50-1000 mg/m.sup.2. In a preferred embodiment, GCSF is administered at between 2 and 70 mcg/kg. In a preferred embodiment, an antiproliferative agent is provided for administration at between about 0.1-50 mg/kg. Generally the concentration of naphthalimide will depend on the NAP-2 genotype of the patient. The dosing schedule is preferably day 1, day 8, day 15 and thereafter at 28 day intervals.

The compositions include compositions suitable for oral, rectal, topical (including transdermal devices, aerosols, creams, ointments, lotions, and dusting powders), parenteral (including subcutaneous, intramuscular, and intravenous), ocular (ophthalmic), pulmonary (nasal or buccal inhalation), or nasal administration; although the most suitable route in any given case will depend largely on the nature and severity of the condition being treated and on the nature of the active ingredient. The agents may be conveniently presented in unit dosage form and prepared by any of the methods well known in the art of pharmacy.

For example, compounds may be administered orally, for example in tablet form, or by inhalation, for example in aerosol or other atomisable formulations or in dry powder formulations, using an appropriate inhalation device such as those known in the art. The compounds of the invention may also be administered intranasally.

In the case of oral delivery, the dosage form would allow that suitable concentrations of a naphthalimide would be provided in a form such that an adequate plasma level could be achieved to provide the chemopotentiation of the other chemotherapeutic compound(s). Tablets, capsules, suspensions or solutions may contain 10 milligrams to 2 grams per dose treatment to achieve the appropriate plasma concentrations.

A compound may be combined as the active ingredient in intimate admixture with a pharmaceutical carrier according to conventional pharmaceutical compounding techniques. The carrier may take a wide variety of forms depending on the nature of the preparation desired for administration, i.e., oral, parenteral, etc. In preparing oral dosage forms, any of the usual pharmaceutical media may be used, such as water, glycols, oils, alcohols, flavoring agents, preservatives, coloring agents, and the like in the case of oral liquid preparations (e.g., suspensions, elixirs, and solutions); or carriers such as starches, sugars, microcrystalline cellulose, diluents, granulating agents, lubricants, binders, disintegrating agents, etc. in the case of oral solid preparations such as powders, capsules, and tablets. Solid oral preparations are preferred over liquid oral preparations. Because of their ease of administration, tablets and capsules are the preferred oral dosage unit form. If desired, capsules may be coated by standard aqueous or non-aqueous techniques.

In addition to the dosage forms described above, the compounds of the invention may be administered by controlled release means and devices.

Pharmaceutical compositions suitable for oral administration may be prepared as discrete units such as capsules, cachets, or tablets each containing a predetermined amount of the active ingredient in powder or granular form or as a solution or suspension in an aqueous or nonaqueous liquid or in an oil-in-water or water-in-oil emulsion. Such compositions may be prepared by any of the methods known in the art of pharmacy. In general, the compositions are prepared by uniformly and intimately admixing the active ingredient with liquid carriers, finely divided solid carriers, or both and then, if necessary, shaping the product into the desired form. For example, a tablet may be prepared by compression or molding, optionally with one or more accessory ingredients. Compressed tablets may be prepared by compressing in a suitable machine the active ingredient in a free-flowing form such as powder or granule optionally mixed with a binder, lubricant, inert diluent, or surface active or dispersing agent. Molded tablets may be made by molding in a suitable machine, a mixture of the powdered compound moistened with an inert liquid diluent.

Ophthalmic inserts are made from compression molded films which are prepared on a Carver Press by subjecting the powdered mixture of active ingredient and HPC to a compression force of 12,000 lb. (gauge) at 149.degree. C. for 1-4 min. The film is cooled under pressure by having cold water circulate in the platen. The inserts are then individually cut from the film with a rod-shaped punch. Each insert is placed in a vial, which is then placed in a humidity cabinet (88% relative humidity at 30.degree. C.) for 2-4 days. After removal from the cabinet, the vials are capped and then autoclaved at 121.degree. C. for 0.5 hr.

The inhalable form may be, for example, an atomisable composition such as an aerosol comprising the compounds of the invention in solution or dispersion in a propellant or a nebulizable composition comprising a dispersion of the compound of the invention in an aqueous, organic or aqueous/organic medium, or a finely divided particulate form comprising the compounds of the invention in finely divided form optionally together with a pharmaceutically acceptable carrier in finely divided form.

The compositions containing a compound of this invention may also comprise an additional agent selected from the group consisting of cortiocosteroids, bronchodilators, antiasthmatics (mast cell stabilizers), anti-inflammatories, antirheumatics, immunosuppressants, antimetabolites, immunomodulators, antipsoriatics, and antidiabetics. Specific compounds include theophylline, sulfasalazine and aminosalicylates (anti-inflammatories); cyclosporin, FK-506, and rapamycin (immunosuppressants); cyclophosphamide and methotrexate (antimetabolites); and interferons (immunomodulators).

An aerosol composition suitable for use as the inhalable form may comprise the compounds of the invention in solution or dispersion in a propellant, which may be chosen from any of the propellants known in the art. Suitable such propellants include hydrocarbons such as n-propane, n-butane or isobutane or mixtures of two or more such hydrocarbons, and halogen-substituted hydrocarbons, for example fluorine-substituted methanes, ethanes, propanes, butanes, cyclopropanes or cyclobutanes, particularly 1,1,1,2-tetrafluoroethane (HFA134a) and heptafluoropropane (HFA227), or mixtures of two or more such halogen-substituted hydrocarbons. Where the compounds of the invention are present in dispersion in the propellant, i.e. where present in particulate form dispersed in the propellant, the aerosol composition may also contain a lubricant and a surfactant, which may be chosen from those lubricants and surfactants known in the art. The aerosol composition may contain up to about 5% by weight, for example 0.002 to 5%, 0.01 to 3%, 0.015 to 2%, 0.1 to 2%, 0.5 to 2% or 0.5 to 1%, by weight of the compounds of the invention, based on the weight of the propellant. Where present, the lubricant and surfactant may be in an amount up to 5% and 0.5% respectively by weight of the aerosol composition. The aerosol composition may also contain ethanol as co-solvent in an amount up to 30% by weight of the composition, particularly for administration from a pressurized metered dose inhalation device.

A finely divided particulate form, i.e. a dry powder, suitable for use as the inhalable form may comprise the compounds of the invention in finely divided particulate form, optionally together with a finely divided particulate carrier, which may be chosen from materials known as carriers in dry powder inhalation compositions, for example saccharides, including monosaccharides, disaccharides and polysaccharides such as arabinose, glucose, fructose, ribose, mannose, sucrose, lactose, maltose, starches or dextran. As especially preferred carrier is lactose. The dry powder may be in capsules of gelatin or plastic, or in blisters, for use in a dry powder inhalation device, preferably in dosage units of 5 .mu.g to 40 mg of the active ingredient. Alternatively, the dry powder may be contained as a reservoir in a multi-dose dry powder inhalation device.

In the finely divided particulate form, and in the aerosol composition where the compounds are present in particulate form, the compound may have an average particle diameter of up to about 10 nanometers, for example 1 to 5 nanometers. The particle size of the compound of the invention, and that of a solid carrier where present in dry powder compositions, can be reduced to the desired level by conventional methods, for example by grinding in an air-jet mill, ball mill or vibrator mill, microprecipitation, spray-drying, lyophilisation or recrystallisation from supercritical media.

The inhalable medicament comprising the pharmaceutical compositions of the invention may be administered using an inhalation device suitable for the inhalable form, such devices being well known in the art. Accordingly, the invention also provides a pharmaceutical product comprising the compounds of the invention in inhalable form as hereinbefore described in association with an inhalation device. In a further aspect, the invention provides an inhalation device containing the compounds of the invention in inhalable form as hereinbefore described.

Where the inhalable form is an aerosol composition, the inhalation device may be an aerosol vial provided with a valve adapted to deliver a metered dose, such as 10 to 100 .mu.1, e.g. 25 to 50 .mu.1, of the composition, i.e. a device known as a metered dose inhaler. Suitable such aerosol vials and procedures for containing within them aerosol compositions under pressure are well known to those skilled in the art of inhalation therapy. Where the inhalable form is a nebulizable aqueous, organic or aqueous/organic dispersion, the inhalation device may be a known nebulizer, for example a conventional pneumatic nebulizer such as an airjet nebulizer, or an ultrasonic nebulizer, which may contain, for example, from 1 to 50 mL, commonly 1 to 10 mL, of the dispersion; or a hand-held nebulizer such as an AERX (ex Aradigm, US) or BINEB (Boehringer Ingelheim) nebulizer which allows much smaller nebulized volumes, e.g. 10 to 100.mu.1, than conventional nebulizers. Where the inhalable form is the finely divided particulate form, the inhalation device may be, for example, a dry powder inhalation device adapted to deliver dry powder from a capsule or blister containing a dosage unit of the dry powder or a multidose dry powder inhalation device adapted to deliver, for example, 25 mg of dry powder per actuation. Suitable such dry powder inhalation devices are well known.

The pharmaceutical compositions of the invention may be synthesized using known techniques. According to one embodiment, the naphthalimide used in the present invention is amonafide synthesized according to a method disclosed in U.S. Publication No. 2004/0082788, published Apr. 29, 2004, hereby incorporated by reference in its entirety.

Nitro-naphthalimide, or a “mitonafide analog”, as indicated in the structure in FIG. 2 is made by adding an aliphatic diamine to 3-nitro-1,8,-naphthalic anhydride in an organic solvent mixture, and refluxing to obtain the nitro naphthalimide. A general scheme depicting the reaction is below:

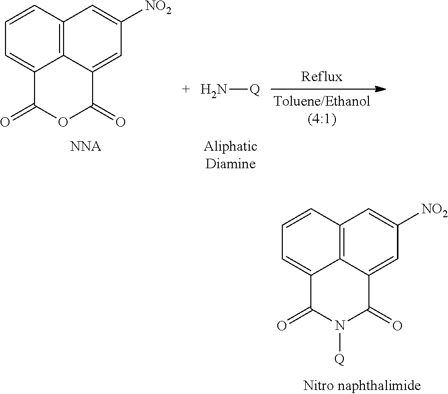

The aliphatic diamine used in the synthesis of a nitro naphthalimide can vary. The choice of aliphatic diamine allows synthesis of a mitonafide analog with, for example, a carbon chain length of 1-6. According to a preferred embodiment, the aliphatic diamine used is N,N-dimethylethylenediamine.

The structure in FIG. 2 indicates a substituent group, Q. Q may represent a variety of structures, including those indicated in FIGS. 4 and 5.

One class of compounds synthesized by the methods of this invention includes naphthalimides with the structure depicted in FIG. 3. The group Q in FIG. 3 may be a variety of substituents, for example, the groups represented in FIG. 4. For example, Q may be 1-R′-azetid-3-yl (FIG. 4a), 1-R′-pyrrolid-3-yl (FIG. 4b), 1-R′-piperid-4-yl (FIG. 4c), 1,2-diR′-1,2-diazolid-4-yl (FIG. 4d), 1,2-diazol-1-en-4yl (FIG. 4e), 1-R′-piperid-3-yl (FIG. 4f), 3-R′-oxazolid-5-yl (FIG. 4g). In FIG. 4, R′=alkyl, unsaturated alkyl, acyl, alkoxy, aryl, amino, substituted amino, sulfo, sulfamoyl, carboxy, carbamyl, cyano.

In another embodiment, the structure in FIG. 3 represents a naphthalimide of the invention, wherein Q represents—(CH.sub.2).sub.2NR.sub.2, wherein R=methyl, ethyl, propyl, butyl, etc. NR.sub.2 in this representation may represent a heterocyclic group. Thus, Q may be any one of the groups shown in FIG. 5

In some embodiments, R.sub.2=-(CH.sub.2).sub.n-where n=2 to 6, or R.sub.2=-(CH.sub.2)mX—(CH.sub.2).sub.n-where m and n can be 0 to 5 and X can be NR″ (where R″=hydrogen, alkyl, unsaturated alkyl, acyl, alkoxy, aryl, amino, substituted amino, sulfo, sulfamoyl, carboxy, carbamyl, cyano, or is not present), O, or S. Furthermore, these cyclic groups may have unsaturated bonds and may also bear substituents such as alkyl, aryl, or heteroaryl.

Further examples of the substituent group Q include, for example, those shown in FIG. 5, which are 1-pyrrolidyl (5a), 3-R′-1-piperidyl (FIG. 5b), morpholino (FIG. 5c), 1-R′-piperazin-4-yl (FIG. 5d), 1-pyrrolyl (FIG. 5e), 1-imidazolyl (FIG. 5f), 1,3,5-triazol-1-yl (FIG. 5g), N-maleimido (FIG. 5h), 2-(R′-imino)pyrrolidyl (FIG. 5i), pyrazin-2-on-1-yl (FIG. 5j), 3-oxazolidyl (FIG. 5k), 3-oxazolyl (FIG. 51), and others known in the art, for example, 2-pyrrolyl, 3-chloro-1-pyrrolidyl, 2-nitro-1-imidazolyl, 4-methoxy-1-imidazolyl, 3-methyl-1-imidazolyl. In the structures depicted in FIG. 5, R′=alkyl, unsaturated alkyl, acyl, alkoxy, aryl, amino, substituted amino, sulfo, sulfamoyl, carboxy, carbamyl, cyano, and other functional groups known to those skilled in the art

Another group of compounds of the invention are naphthalimides having an amino group attached to other positions in the naphthalimide rings. According to one embodiment, the naphthalimide ring is modified to include one or more amino groups at positions other than position 3 of the naphthalimide ring. According to another embodiment, the naphthalimide ring is modified to include one or more amino groups at positions in addition to the amino group at position 3 of the naphthalimide ring. In another embodiment, the amino group at position 3 is replaced with a substituent group. Examples of such groups include: alkyl, aryl, nitro, substituted amino, sulfamoyl, halo, carboxy, carbamyl, cyano, and other functional groups known to those skilled in the art. In yet another embodiment, an additional group is attached to the naphthalimide ring also comprising an amino group at position 3. Examples of such substituent groups include: alkyl, aryl, nitro, amino, substituted amino, sulfamoyl, halo, carboxy, carbamyl, cyano, and other functional groups known to those skilled in the art

Alternatively, the amino group at position 3 may be replaced by other substituent groups. Examples of substituent groups include: alkyl, aryl, nitro, substituted amino, sulfamoyl, halo, carboxy, carbamyl, cyano, and other functional groups known to those skilled in the art

The naphthalene ring can be replaced with one bearing one or more nitrogen atoms in either or both rings. An example would be isoquinoline analogs (FIG. 6), where Q is as previously defined. A preferred isoquinoline analog of amonafide is where Q is-(CH.sub.2)n-N(CH.sub.3)-.sub.2, where n is 1-12 or more. In a more preferred embodiment, n is 1-6. The isoquinoline analog may also have one or more substituent groups (as described herein for other analogs) reducing one or more hydrogens of the methyl and/or methylene groups

An organic solvent is used in the method of the invention for refluxing the aliphatic diamine and 3-nitro-1,8,-naphthalic anhydride. In one embodiment, the organic solvent is ethanol. In another embodiment, the organic solvent is dimethylformamide. In yet another embodiment, the organic solvent is toluene-ethanol. In a preferred embodiment, the organic solvent is toluene-ethanol in a 4:1 ratio

The mixture is refluxed and monitored, for example, by thin-layer chromatography. Refluxing is performed according to one embodiment for 30 minutes. The resulting mixture is filtered and evaporated to obtain a brown solid of mitonafide or a mitonafide analog

Each of these naphthalimides may be converted into a mono or diammonium salt as discussed infra.

ii. Naphthalimides

According to another embodiment, the invention includes a method of synthesis of a naphthalimide. In a preferred embodiment, the naphthalimide is amonafide (See Example 2). Amonafide is also known as 5-amino-2-[(dimethylamine)ethyl]-1H-benz[de-]disoquinoline-1,3-(2H)-dione.

The method of naphthalimide synthesis involves dissolving mitonafide or a mitonafide analog in an organic solvent. The organic solvent, according to one embodiment, is dichloromethane-methanol. In a preferred embodiment, dichloromethane-methanol is used in a ratio of 4:1 at 25 mL/g mitonafide

The method of naphthalimide synthesis further involves adding a reducing agent (e.g., ammonium formate) to the dissolved mitonafide or mitonafide analog together with a catalyst. A variety of reducing agents suitable for reduction of the 3-nitro group are known in the art, including hydrazine, tetralin, ethanol, ascorbic acid, formic acid, formate salts, and phosphinic acid (see, e.g., Johnstone, R. A. W. et al., Chemical Reviews 85 (2) 129 (1985); Entwhistle, I. D. et al., J. C. Soc. Perkin Trans. 1,443 (1977)). According to a preferred embodiment, the reducing agent is ammonium formate. Other formate salts include substituted ammonium formates such as 2-hydroxyethylmethyl ammonium formate, methyl ammonium formate and morpholinium. According to a preferred embodiment, 4.5 mol equivalents of ammonium formate are used.

The method of naphthalimide synthesis involves use of a catalyst. A variety of suitable catalysts are known in the art, including the noble metals Pd, Pt, Rh and Raney Nickel (see, e.g., Johnstone, R. A. W. et al. (1985), supra, and Entwhistle, I. D. et al. (1977), supra). In one embodiment, the catalyst is palladium-carbon. In a preferred embodiment, 10% palladium-carbon (about 20% mitonafide weight) is added. The catalyst is mixed at room temperature under nitrogen for about 1 hour. The method further involves filtering the mixture and adding the mixture to a cool water bath (<10.degree. C.) to precipitate. After filtration, a precipitate forms which is dried to yield a naphthalimide, for example, amonafide

### (C. sub.16H.sub.17N.sub.3O.sub.2).

iii. Naphthalimide Salts

A further embodiment of the invention includes methods of synthesis of naphthalimide diammonium salts.

In general, naphthalimides are dibasic compounds containing at least two amines and in most cases an amine group covalently linked to an aromatic group. When in contact with an acid, at least one or two of the amines within the naphthalimide may be protonated by reaction with an inorganic or an organic acid to form salts. Such salts are generally weak acids comprising primary, secondary or tertiary ammonium ions formed by protonation of an amine within the amonifide molecule. The counter-ions for such ammonium ions can be any appropriate anion capable of being used in a pharmaceutical composition. In some embodiments, the acidic salts are formed by reacting the naphthalimide with a mineral (inorganic) or organic acid. Such mineral acids include hydrochloric acid, hydrobromic, acid, sulfuric acid, nitric acid and phosphoric acid. Some organic acids which may be used in forming salts of modified include acetic acid, proprionic acid, glycolic acid, pyruvic acid, oxalic acid, maleic acid, malic acid, malonic acid, succinic acid, hydroxy succinic acid, fumaric acid, tartaric acid, citric acid, benzoic acid, cinnamic acid, mandelic acid, methanesulfonic acid, ethanesulfonic acid, p-toluenesulfonic acid and salicylic acid. Inorganic acids include hydrochloric acid, hydrobromic, acid, sulfuric acid, nitric acid and phosphoric acid.

In most embodiments, two amines in the naphthalimide will be protonated to form a diammonium salt. In preferred embodiments, at least 1.5, more preferably 1.75, still more preferably 1.9, still more preferably 1.95, still more preferably 1.99, and most preferably 2.0 mol equivalents of the two amines in the amonafide are protonated. In some instances where more than two amines are present, the possibility exists for protonation of all, or a portion of all, of the amines. For example, if three amines are present, least 1.5, more preferably 1.75, still more preferably 1.9, still more preferably 2.0, still more preferably 2.5, still more preferably 2.75, still more preferably 2.9, still more preferably 2.95, still more preferably 2.99, and most preferably 3.0 mol equivalents of the three amines are protonated.

In one embodiment, the amines of the naphthalimide have similar pK's for protonation. Upon titration of the free base amines of this embodiment with an acid, the amines are similarly protonated during the range of titration. In a preferred embodiment, at least two of the amines of the naphthalimide are greater than 50% protonated, more preferably greater than 75% protonated, still more preferably greater than 90% protonated, still more preferably greater than 95% protonated, still more preferably greater than 99% protonated, and most preferably 100% protonated.

In a further embodiment of the invention, the amines of the naphthalimide have different pK's for protonation. Upon titration of the amines with an acid, the amines will become protonated in a multiphasic manner according to their pK's. For example, the amine that has a higher pK value will become protonated before the amine that has a lower pK value when the free acid form of the naphthalimide is titrated with an acid. In a preferred embodiment, at least one of the amines of the naphthalimide is protonated and at least one of the amines of the naphthalimide is subsequently protonated, preferably greater than 50% protonated, more preferably greater than 75% protonated, still more preferably greater than 85% protonated, still more preferably greater than 95% protonated, still more preferably greater than 99% protonated and most preferably 100% protonated.

Diammonium salts of naphthalimide generally refers to naphthalimide salts which contain two protonated amines with the naphthalimide structure. Partial diammonium salts include those naphthalimides wherein at least 1.5 mol equivalents of the amines are protonated. In some embodiments, the counter-ions may be a mixture of one or more of the base forms of the aforementioned inorganic and/or organic acids.

In a preferred embodiment, the naphthalimide diammonium salt is amonafide dihydrochloride

According to this embodiment, HCl gas is bubbled over amonafide solution to precipitate a salt form of amonafide. The process is robust and easy to scale up. Amonafide monohydrochloride as disclosed by U.S. Pat. No. 5,420,137 is manufactured by reaction with calculated amount of HCl solution. This process may result inaccurate amount of HCl in the final product and this process is not easy to scale up

Dihydrochloride salt is more acidic and more soluble in water, as compared to a monohydrochloride salt. As a result, a wider range of drug concentration can be achieved to facilitate further manufacturing process such as lyophilization and more flexible to meet clinical needs.

A preferred embodiment of the present invention provides an improved synthesis of amonafide dihydrochloride salt exhibiting a well-defined crystalline structure with a narrow melting temperature range. The characteristic physical and chemical properties and stability of this form improve the safe handling of this cytotoxic drug during the manufacture of pharmaceutical dosage forms such as oral products including tablet and capsule forms, as well as a wide range of injectable dosage forms, such as liquid or lyophilized forms

The creation of mono and diammonium salt forms enables the generation of pharmaceutically relevant dosages useful for the treatment of aberrant cell conditions such as hyperproliferative diseases, including, for example, cancer and precancerous conditions.

### Pharmaceutical Dosage Forms and Modes of Administration

The National Cancer Institute has conducted clinical trials in cancer chemotherapy using a lyophilized amonafide product. Certain information regarding its chemistry and its pharmaceutical formulation are given in the publication titled AMONAFIDE (NSC-308847), NCI Investigational Drugs, Pharmaceutical data (1994). Notably, the dosage is a sterile 500 mg (as the base) vial. Constitution with 9.6 mL of Sterile Water for Injection, USP or 0.9% Sodium Chloride Injection, USP, results in a solution containing 50 mg/mL of amonafide with pH adjusted to 5 to 7 with sodium hydroxide. Reconstitution can be problematic if improperly performed and is better avoided

One objective of the present invention, therefore, is to provide a stable, therapeutically acceptable, intravenously injectable dosage form of a naphthalimide or naphthalimide salt (e.g., amonafide) that does not require lyophilization and reconstitution, can be packaged and shipped as single vial instead of a dual-vial package, and can be supplied in a liquid formulation from 1-250 mg/mL. These objectives are achieved by the present invention, as described in detail below.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well known in the art of pharmacy

Additives, carriers or excipients are well known in the art, and are used in a variety of formulations. See for example, Gilman, A. G. et al., eds., THE PHARMACOLOGICAL BASIS OF THERAPEUTICS, 8.sup.th Ed. Pergamon Press, New York, (1990), incorporated herein by reference in its entirety. In practical use, the compositions of the invention can be combined as the active ingredient in intimate admixture with a pharmaceutical carrier or excipient according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols (e.g., polyethylene glycol), oils, alcohols, flavoring agents, sweeteners, preservatives, coloring agents and the like in the case of oral liquid preparations, such as, for example, suspensions, elixirs and solutions; or carriers such as starches (e.g. corn or other), sugars, lactose, serum albumin, microcrystalline cellulose, buffers (e.g., sodium acetate), diluents, granulating agents, lubricants, binders, disintegrating agents and the like in the case of oral solid preparations such as, for example, powders, hard and soft capsules and tablets, with the solid oral preparations being preferred over the liquid preparations.

i. Oral Dosage Forms

Because of their ease of administration, tablets and capsules represent a particularly advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

ii. Liquid Dosage Forms

The present invention also includes a liquid dosage form of a naphthalimide or naphthalimide salt prepared according to the methods described herein.

In one embodiment, the liquid dosage form prepared according to the methods of the invention is a stable sterile aqueous solution of a naphthalimide or naphthalimide salt (e.g., amonafide or amonafide dihydrochloride) in a sealed container such as an ampoule or vial, is in unit dosage form suitable for intravenous administration, has a concentration of a naphthalimide or naphthalimide salt between about 1 and about 250 mg/mL, and has a pH between about 3.0 and 7.0. In a preferred embodiment, the concentration of a naphthalimide or naphthalimide salt is about 20 mg/mL.

In a preferred embodiment, the pH of the liquid dosage form is about 6.0. Preferably, the pH is adjusted, if necessary, using a nontoxic, pharmaceutically and therapeutically acceptable inorganic source base. In a preferred embodiment, the base is a mineral base. In a more preferred embodiment, the base is sodium hydroxide.

In one embodiment, the liquid dosage form prepared according to the methods of the invention preferably is free of any other added chemicals. In another embodiment, the liquid dosage form contains a customary, physiologically acceptable excipient or carrier such as a preservative or tonicity agent. In one embodiment, an aqueous solution of amonafide comprises a carrier or excipient. Preferably, a carrier or excipient, when provided, is present at a concentration between about 0.1 mg/ml to 100 mg/ml

According to one embodiment, the liquid dosage form is stable. “Stable” means that the liquid dosage form exhibits less than 5% loss of potency as measured by high performance liquid chromatography (HPLC) upon storage for 1 month at 60.degree. C. or 9 months 40.degree. C.

The compositions of the invention may be conveniently presented in unit dosage forms, and prepared by any methods known in the art of pharmacy. The term “unit dosage form” refers to physically discrete units suitable as unitary dosages for human subjects and other mammals, each unit containing a predetermined quantity of active material calculated to produce the desired therapeutic effect in association with a suitable pharmaceutical excipient or carrier

In the preferred embodiment, the pharmaceutical compositions are water soluble, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

### Example I

This example describes patient genotyping to improve dosing of naphthalimide to reduce potential toxic side effects

Thirty-two (32) patients were treated amonafide dihydrochloride. Sixteen (16) were male and sixteen (16) were female. The mean age was 65 years. The dose schedule was days 1, 8, 15 and thereafter every 28 days. The dosages ranged from 300 to 500 mg/m.sup.2 in a dose escalation studied to determine dose limiting toxicity (DLT).

Patients were genotyped to determine their NAT-2 (N-acetyl transferase) genotype. See D. W. Hein, et al., “Molecular Genetics and Epidemiology of the NAT1 and NAT2 Acetylation Polymorphisms,” Cancer Epidemiology, Biomarkers & Prevention Vol. 9, 29-42, January 2000. Fourteen (14) patients had the slow acylator genotype whereas eighteen (18) patients were fast acylators having either the rapid (liomozygous) or intermediate (heterozygous) genotype.

Serum levels of amonafide and acetyl amonafide were determined by HPLC. A significant difference was observed in the conversion of amonafide (AMF) to N-acetyl amonafide (AAMF) for patients within the different genotype groups.

For slow acylators, the mean ratio AAMF to AMF was 0.65 (range of 0.28-1.131), as measured by concentrations X time.

For slow acylators, the mean ratio AAMF to AMF was 0.65 (range of 0.28-1.131), as measured by concentrations X time.

In the case of fast acylators, the mean ratio of AAMF to AMF was 2.75 with a range of 1.2-5.98.

Myelosuppression was correlated with the genotype of the patient.

TABLE 2

| | | Average % |
|---|---|---|
| | Dose | Reduction of |
| Genotype | (mg/m · sup · 2) | Neutrophils |
| I/R (Fast) | 400 | 78 |
| (18 patients) | | |
| S (Slow) | 500 | 55 |
| (14 patients) | | |

As can be seen, slow acylators dosed at 500 mg/m.sup.2 presented with a 55% reduction in neutrophils. Fast acylators dosed with lower amounts of amonafide (400 mg/m.sup.2) had a neutrophil reduction of almost 80%.

Based on such results, fast acylators can be dosed at lower levels of naphthalimide and appropriate doses of GCSF. Alternatively, the dose of the naphthalimide for the fast acylators can be increased with corresponding increase in the dosing of GCSF.

In the case of the slow acylators, the physician has the option not to administer GCSF or to administer GCSF at a conservative dose level.

We claim:

1. A method for dosing a patient with a naphthalimide comprising:
genotyping a patient for an N-acetyl transferase genotype to provide an indication of the phenotype of said patient to rapidly or slowly acylate a naphthalimide; and
determining the dose of napthalimide to be administered to said patient.

2. A method for dosing a patient with a naphthalimide comprising:
genotyping a patient for an N-acetyl transferase genotype to provide an indication of the phenotype of said patient to rapidly or slowly acylate a naphthalimide; and
administrating a naphthalimide to said patient at a dose that is dependent upon the acylation phenotype.

3. The method of claim 1 or 2 wherein said naphthalimide is amonafide.

4. The method of claim 2 wherein a higher amount of naphthalimide is administered to a patient with a slow acylation phenotype as compared to a fast acylator phenotype.

5. The method of claim 2 wherein a lower dose of the naphthalimide is administered to a patient with a fast acylation phenotype as compared to a slow acylator phenotype.

6. The method of claim 1 further comprising determining the dose of granulocyte colony stimulating factor (GCSF) to be administered to said patient based on the determination of said fast or slow acylation phenotype to treat leukocytopenia associated with administration of said naphthalimide.

7. The method of claim 2 further comprising administering a dose of granulocyte colony stimulating factor (GCSF) based on the determination of said fast or slow acylation phenotype to treat leukocytopenia associated with administration of said naphthalimide.

8. The method of claim 7 wherein said GCSF is administered contemporaneously with said naphthalimide.

9. The method of claim 7 wherein said GCSM is administered prior to said naphthalimide.

10. The method of claim 7 wherein said GSCSF is administered after the administration of said naphthalimide.

11. The method of claim 7 further comprising administering an anti-proliferative agent to said patient before, during or after the administration of either of said naphthalimide or said GSCSF.

12. A composition comprising a naphthalimide and GCSF.

13. A composition according to claim 12 wherein said naphthalimide is amonafide.

14. The composition of claim 12 further comprising anti-proliferative agent.

15. A kit for N-acetyl transferase genotyping comprising:
at least one nucleic acid capable of distinguishing at least one allele of a genotype of N-acetyl transferase; and
at least one reagent or label required for said genotyping.

16. The kit of claim 15 wherein one or more reagents include a DNA polymerase for carrying out a polymerase chain reaction (PCR).

17. A kit comprising naphthalimide and GCSF.

18. The kit of claim 17 further comprising an anti-proliferative agent.
